(19) United States
(12) Patent Application Publication
(10) Pub. No.: US 2012/0108974 A1
(43) Pub. Date: May 3, 2012

(54) ULTRASOUND DIAGNOSTIC APPARATUS AND PROGRAM

(75) Inventor: Yoshiki KATOU, Tokyo (JP)

(73) Assignee: KONICA MINOLTA MEDICAL & GRAPHIC, INC., Tokyo (JP)

(21) Appl. No.: 13/280,540

(22) Filed: Oct. 25, 2011

(30) Foreign Application Priority Data
Oct. 28, 2010 (JP) JP2010-242152
May 20, 2011 (JP) JP2011-113053

(51) Int. Cl. A61B 8/00

(52) U.S. Cl. 600/445

(57) ABSTRACT

An ultrasound diagnostic apparatus includes: an ultrasound probe; a transmission section which supplies the probe with a drive signal; a receiving section which receives a reception signal sent from the probe; an image generation section which generates frame image data obtained by converting the reception signal; an intermediate image generation section which detects a moved image, identifies a move source and a move destination of the detected image based on a plurality of frame image data, and generates intermediate image data which allocates the moved image at a position obtained by intermediating the identified move source and the identified move destination of the detected image; and a display section which displays the ultrasound diagnosis image, wherein the display section displays the ultrasound diagnosis image so that intermediate image is inserted and displayed in a chronological order into image based on the frame image data.

### CROSS REFERENCE TO RELATED APPLICATION

The present application is based on Japanese Patent Application No. 2010-242152 filed with Japanese Patent Office on Oct. 28, 2010 and Japanese Patent Application No. 2011-113053 filed with Japanese Patent Office on May 20, 2011, the entire content of which is hereby incorporated by reference.

### FIELD OF THE INVENTION

The present invention relates to an ultrasound diagnostic apparatus and program.

### BACKGROUND

What is commonly known in the prior art includes an ultrasound diagnostic apparatus equipped with a vibration probe made up of an array of multiple transducers, wherein ultrasound waves are exchanged with a subject such as a living body, and ultrasound image data is generated on a per-frame basis based on the data obtained from the received ultrasound wave and is displayed on an image display device.

Most of the display devices used in the ultrasound diagnostic apparatus in recent years are liquid crystal displays. A hold type display, for example, is often used as the liquid crystal display. In such a hold type liquid crystal display, emissions in part of the past frame and that of the current frame are integrated so that a residual image is issued when a moving object is displayed.

By contrast, in one of the conventional ultrasound diagnostic apparatuses, an impulse drive type display is used to restrict the pixel emission period in one frame to a prescribed period, thereby suppressing the residual image (e.g., Japanese Unexamined Patent Application Publication No. 2010-46343).

### Problems to be Solved by the Invention

Incidentally, to get a more accurate ultrasound image in an ultrasound diagnostic apparatus, it is necessary to increase the number of transmissions and receptions of ultrasound waves in one frame and to prolong the time for each transmission and reception. This causes the frame rate to drop, and if the image moves, the motion will be less smooth and the object of diagnosis will go out of sight. This can disturb accurate diagnosis.

The ultrasound diagnostic apparatus disclosed in the aforementioned Japanese Unexamined Patent Application Publication No. 2010-46343, however, fails to solve the problem of frame rate drop, although the residual image can be suppressed.

### SUMMARY OF THE INVENTION

One aspect of the present invention is an ultrasound diagnostic apparatus comprising: an ultrasound probe including a transducer which outputs ultrasound waves toward a subject by means of a drive signal and outputs a reception signal by receiving the ultrasound waves reflected from the subject; a transmission section which supplies the transducer with the drive signal; a receiving section which receives the reception signal sent from the transducer; an image generation section which generates frame image data obtained by converting the reception signal received by the receiving section, into brightness information showing brightness of image; an intermediate image generation section which detects a moved image out of a plurality of frame image data in different frames, generated by the image generation section, identifies a move source and a move destination of the detected moved image based on the plurality of frame image data, and generates intermediate image data which allocates the moved image at a position obtained by interpolating the identified move source and the identified move destination of the detected moved image; and a display section which displays the ultrasound diagnosis image, based on the frame image data generated by the image generation section and the intermediate image data generated by the intermediate image generation section, wherein the display section displays the ultrasound diagnosis image so that intermediate image based on the intermediate image data generated by the intermediate image generation section is inserted in a chronological order into image based on the frame image data generated by the image generation section.

Another aspect of the present invention is a computer-readable recording medium storing a program to be executed by a computer provided in an ultrasound diagnosis apparatus including a transducer which outputs ultrasound waves toward a subject by means of a drive signal and outputs a reception signal by receiving ultrasound waves reflected from the subject, wherein the program makes the computer function as: a transmission section which supplies the transducer with a drive signal; a receiving section which receives the reception signal output from the transducer, an image generation section which generates frame image data obtained by converting the reception signal received by the receiving section, into brightness information showing a brightness of the image; an intermediate image generation section which detects moved image out of a plurality of frame image data in different frames, generated by the image generation section and, based on the plurality of frame image data, identifies positions of a source and a destination of the detected image, and generates intermediate image data which allocates a moved image at a position obtained by interpolating positions of an origin and a destination of movement of the detected image; and a display section which displays the ultrasound diagnosis image, based on the frame image data generated by the image generation section and the intermediate image data generated by the intermediate image generation section, wherein the ultrasound diagnosis image is displayed on the display section so that intermediate image based on the intermediate image data generated by the intermediate image generation section is inserted in a chronological order into image based on the frame image data generated by the image generation section.

### BRIEF DESCRIPTION OF THE DRAWINGS

FIG. 1 is a diagram representing the external structure of an ultrasound diagnostic apparatus in an embodiment of the present invention;

FIG. 2 is a block diagram representing the approximate structure of the ultrasound diagnostic apparatus;

FIG. 3 is a block diagram representing the functional structure of the intermediate image generation section;

FIGS. 4a and 4b is a diagram showing the motion vector detection technique;

FIGS. 5a and 5b are diagrams showing the motion vector detection technique;

FIG. 6 is a diagram showing the block image structure;

FIGS. 7a, 7b and 7c are diagrams showing the generation of intermediate image data;

FIGS. 8a, 8b and 8c are diagrams showing the generation of intermediate image data;

FIG. 9 is a diagram showing the process of setting the number of pieces of intermediate image data;

FIG. 10 is a diagram showing the setting of the number of pieces of intermediate image data according to frame rate;

FIG. 11 is a diagram showing the intermediate image data generation process;

FIGS. 12a, 12b and 12c are diagrams showing the storage of image data;

FIG. 13 is a diagram showing the freeze control process; and

FIG. 14 is a diagram showing the freeze control process.

### DESCRIPTION OF EMBODIMENTS

The following describes the ultrasound diagnostic apparatus in the present embodiment of the present invention with reference to drawings, without the scope of the invention being restricted to the illustrated examples. The portions having the same function and structure will be assigned the same numerals of reference, and will not be described to avoid duplication.

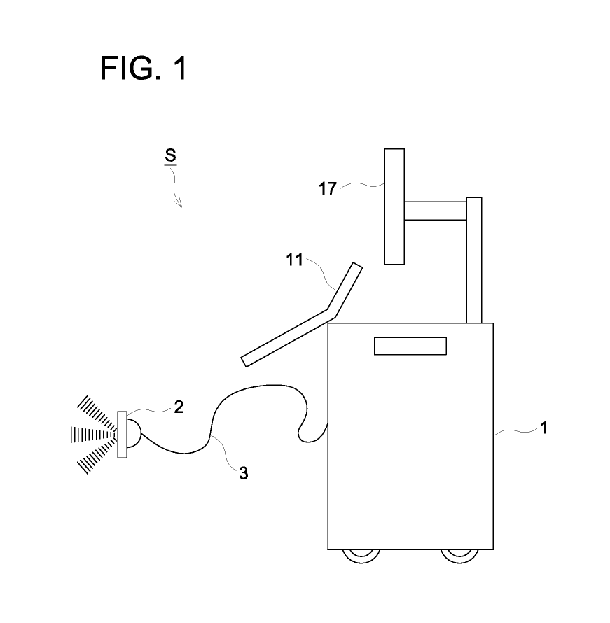

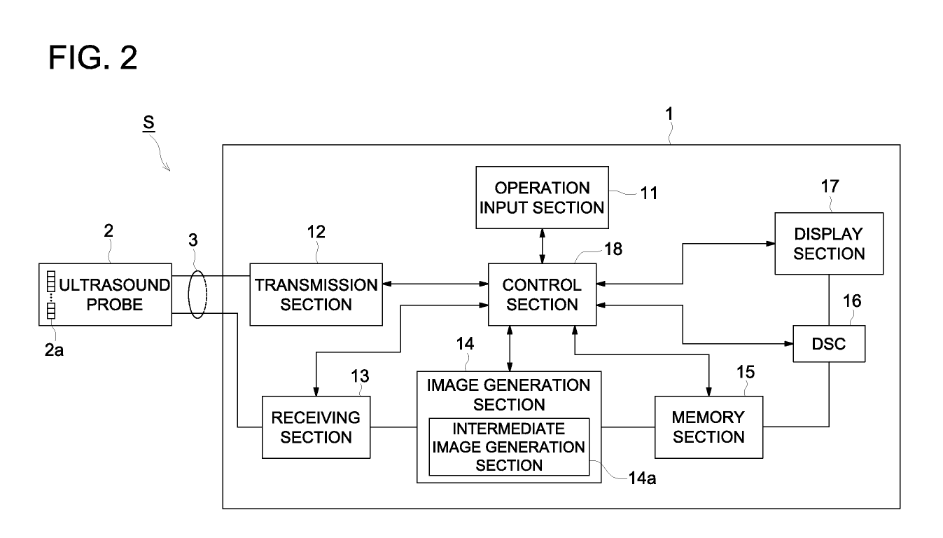

As shown in FIGS. 1 and 2, the ultrasound diagnostic apparatus S as an embodiment of the present invention includes an ultrasound diagnostic apparatus main body 1 and an ultrasound probe 2. The ultrasound probe 2 sends ultrasound waves (for transmission) to such a subject as a living body (not illustrated) and receives the reflected ultrasound waves (echo) reflected from this subject. The ultrasound diagnostic apparatus main body 1 is connected with the ultrasound probe 2 through a cable 3, and sends an electric drive signal to the ultrasound probe 2, thereby allowing the ultrasound probe 2 to send transmission ultrasound waves to the subject At the same time, based on the reception signal as an electric signal generated by the ultrasound probe 2 according to the reflected ultrasound waves from inside the subject received by the ultrasound probe 2, the ultrasound diagnostic apparatus main body 1 converts the internal state of the subject into an ultrasound image.

The ultrasound probe 2 is equipped with a transducers 2a consisting of a piezoelectric element. A plurality of transducers 2a are arranged in a one-dimensional array, for example, in the direction of orientation (in the scanning direction or vertical direction). The present embodiment uses an ultrasound probe 2 provided with n(e.g., 192)-transducers 2a. The transducers 2a can be arranged in a two-dimensional array. The number of the transducers 2a can be determined as desired. In the present embodiment, a linear electronic scanning probe is used as an ultrasound probe 2. Any one of the electronic scanning method and mechanical scanning method can be used. Further, any of the linear scanning method, sector scanning method and convex scanning method can be adopted.

The ultrasound diagnostic apparatus main body 1 includes an operation input section 11, transmission section 12, receiving section 13, image generation section 14, memory section 15, DSC (Digital Scan Converter) 16, display section 17 and control section 18, for example, as shown in FIG. 2.

The operation input section 11 includes various switches, buttons, track ball, mouse and keyboard for entering the diagnosis start instruction command, private information of a subject and other data as well as the input data for freeze operation and frame advance operation. The operation signal is sent to the control section 18. The number of intermediate images to be inserted (described later) can be set by the input operation of the operation input section 11.

Under control of the control section 18, the transmission section 12 supplies the electric drive signal to the ultrasound probe 2 through the cable 3, and allows the ultrasound probe 2 to generate transmission ultrasound waves. Further, the transmission section 12 is equipped with a clock generation circuit, delay circuit and pulse generation circuit, for example. The clock generation circuit generates clock signals for determining the drive signal transmission timing and transmission frequency. The delay circuit sets the delay time of the drive signal transmission timing for each path corresponding to each transducer 2a so that the drive signal is delayed by the preset delay time and the transmission beam made up of transmission ultrasound waves is converged. The pulse generation circuit generates pulse signals as drive signals at a prescribed period.

Under the control of the control section 18, the receiving section 13 receives the electric reception signal from the ultrasound probe 2 through the cable 3. The receiving section 13 is equipped with an amplifier, analog-to-digital conversion circuit, and phased adding circuit, for example. The amplifier amplifies the reception signal at a prescribed amplification rate having been preset, for each path corresponding to each transducer 2a. The analog-to-digital conversion circuit applies analog-to-digital conversion to the amplified reception signal. The phasing adding circuit assigns delay time to the reception signal subjected to analog-to-digital conversion to adjust temporal phase for each path corresponding to each transducers 2a The resulting data is added (for phasing) to generate sound ray data.

The image generation section 14 applies processing of logarithmic amplification or enveloping wave detection to the sound ray data coming from the receiving section 13, thereby generating B-mode image data The B-mode image data represents the intensity of the reception signal in terms of brightness. The image generation section 14 is equipped with an intermediate image generation section 14a.

The intermediate image generation section 14a inputs two frames of the B-mode image data generated in the aforementioned procedure by the image generation section 14. Then, one or more pieces of intermediate image data are generated from the inputted two frames of B-mode image data This intermediate image data to be described in details later is used to insert images in chronological order between the images displayed in conformity to the inputted two frames of B-mode image data. The B-mode image data structured for each frame may be called the frame image data

The B-mode image data and intermediate image data generated in the aforementioned procedure are sent to the memory section 15.

The memory section 15 is composed of a semiconductor memory such as a DRAM (Dynamic Random Access Memory), and stores the B-mode image data sent from the image generation section 14 in units of frames. To be more specific, such data can be stored as frame image data Further, the memory section 15 stores the intermediate image data sent from the image generation section 14. The stored frame image data and intermediate image data are sent to the DSC 16 under the control of the control section 18.

The DSC 16 converts the frame image data and intermediate image data received from the memory section 15, into the image signal by the TV signal scanning method, and the image signal is output to the display section 17.

Such a display device as an LCD (Liquid Crystal Display), CRT (Cathode-Ray Tube) display, organic EL (Electronic Luminescence) display, inorganic EL display and plasma display can be used as the display section 17. The present embodiment is effective particularly when applied to the ultrasound diagnostic apparatus using the LCD or organic EL. The display section 17 displays an image on the display screen in conformity to the image signal sent from the DSC 16. Instead of the display device, a printing device such as a printer can be used.

The control section 18 includes a CPU (Central Processing Unit), ROM (Read Only Memory), and RAM (Random Access Memory), for example. The control section 18 reads various processing programs such as system programs stored in the ROM, and develops them on the RAM, and provides centralized control of the operation of each part of the ultrasound diagnostic apparatus S according to the developed program. The ROM is made of such a nonvolatile memory as semiconductor and stores the system program compatible with the ultrasound diagnostic apparatus S and various processing programs running on this system program. These programs are stored in the computer-readable code format and the CPU sequentially performs the operations in conformity to the relevant program code. The RAM serves as a work area for temporarily storing the programs executed by the CPU.

The following describes how the control section 18 allows the memory section 15 to store the frame image data generated by the image generation section 14 and the intermediate image data generated by the intermediate image generation section 14a. The control section 18 permits the memory section 15 to store the frame image data generated by the image generation section 14 and the intermediate image data generated by the intermediate image generation section 14a each in the format that can be identified.

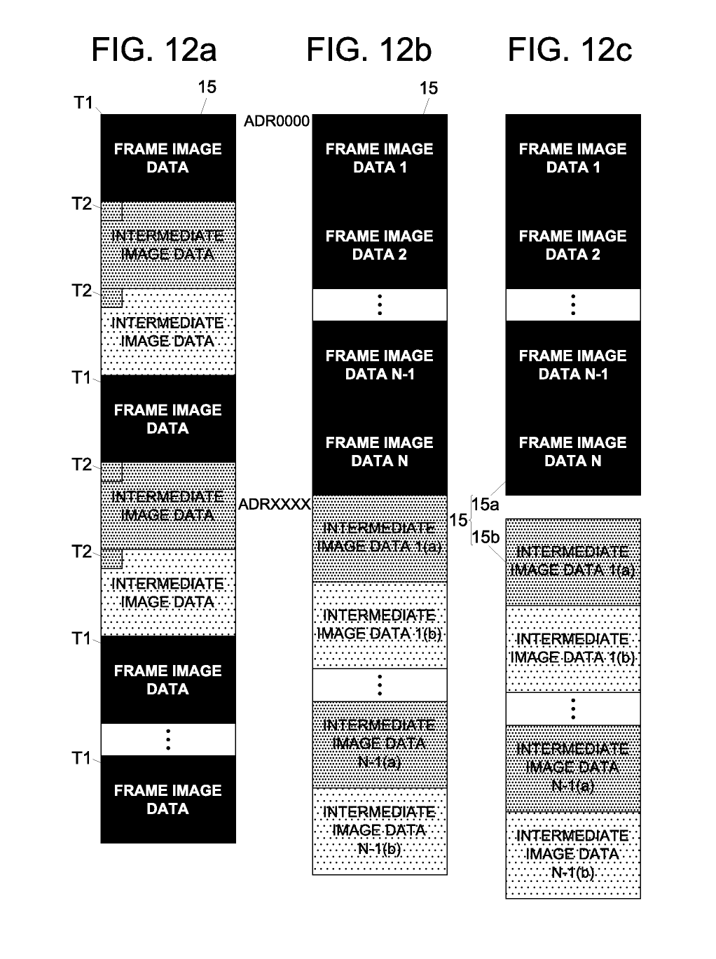

To be more specific, as shown in FIG. 12a, when the frame image data generated by the image generation section 14 is stored in the memory section 15, the control section 18 assigns frame image data with an identification information (tag information) T1 for identifying the frame image data. Similarly, when the intermediate image data generated by the intermediate image generation section 14a is stored in the memory section 15, the control section 18 assigns intermediate image data with the identification information (tag information) T2 for identifying the intermediate image data. This procedure makes it possible to determine if the image data stored in the memory section 15 is the frame image data generated by the image generation section 14 or the intermediate image data generated by the intermediate image generation section 14a. It is also possible to make such arrangements that the control section 18 assigns the identification information to either the frame image data or intermediate image data so that, based on the presence or absence of the identification information, identification is made to see if the image data is the frame image data or intermediate image data.

As shown in FIG. 12b, the control section 18 can allow different storage regions in a memory section 15 to separately store the frame image data generated by the image generation section 14 and the intermediate image data generated by the intermediate image generation section 14a. To be more specific, the control section 18 can provide control in such a way that the frame image data 1 through frame image data N generated by the image generation section 14 are stored in the region indicated by a prescribed address inside the memory section 15 (e.g., region from ADR0000 through the address immediately before ADRXXXX of FIG. 12b), and the intermediate image data 1 through intermediate image data N-1 generated by the intermediate image generation section 14a are stored in the region indicated by the separate address inside the memory section 1 (e.g., region from the ADRXXXX onward of FIG. 12b).

Further, as shown in FIG. 12c, the control section 18 allows different memory sections to separately store the frame image data generated by the image generation section 14 and the intermediate image data generated by the intermediate image generation section 14a. To be more specific, It is also possible to arrange such a configuration that the memory section 15 is made of a memory section 15a and memory section 15b, and the control section 18 allows one of the memory sections 15a to store the frame image data generated by the image generation section 14, and permits the other memory section 15b to store the intermediate image data generated by the intermediate image generation section 14a.

The following describes the functional structure of the intermediate image generation section 14a.

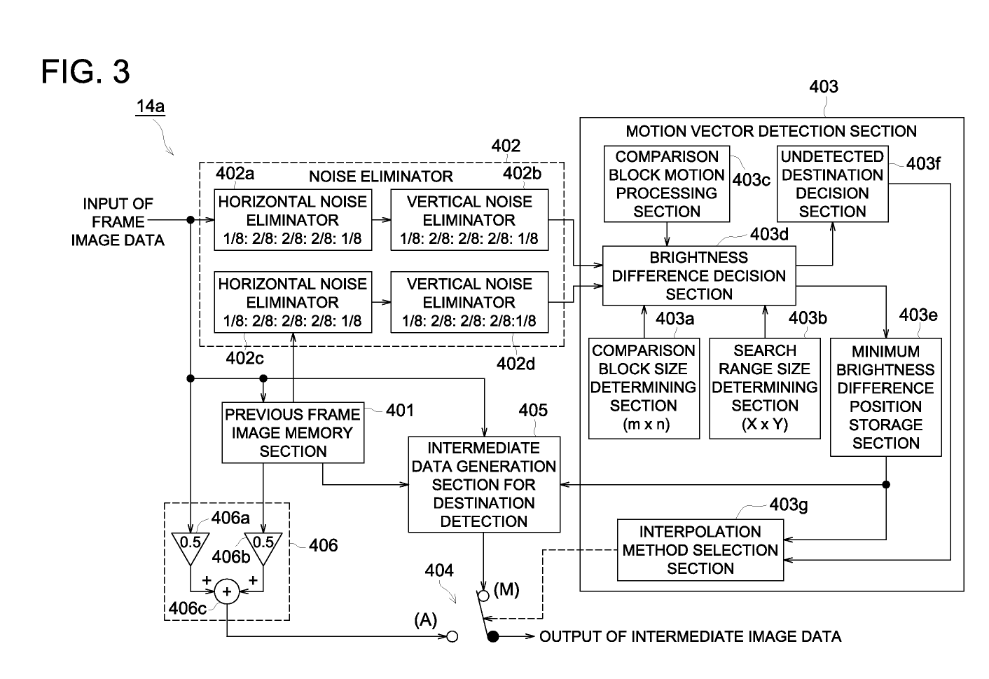

As shown in FIG. 3, the intermediate image generation section 14a includes a previous frame image memory section 401, noise eliminator 402, motion vector detector 403, switching section 404, intermediate data generation section for destination detection 405, and intermediate data generation section 406 when the destination is not detected.

The previous frame image memory section 401 stores the frame image data based on the B-mode image data last generated in the image generation section 14. To be more specific, the previous frame image memory section 401 stores the frame image data generated in the frame one frame before the latest one.

The intermediate image generation section 14a ensures that the B-mode image data generated by the image generation section 14 is held in a buffer (not illustrated) until one frame of B-mode image data is generated. When the frame image data has been generated, namely, when the frame image data of the updated frame has been generated, this frame image data is inputted into the noise eliminator 402. The noise eliminator 402 is provided with a horizontal noise eliminator 402a and vertical noise eliminator 402b. Noise in the horizontal direction is eliminated by the horizontal noise eliminator 402a. The horizontal noise eliminator 402a applies the band limiting filter to smoothen abrupt noise in the horizontal direction. In the present embodiment, an attention pixel is extracted from the inputted frame image data in a prescribed sequence. Centering on this attention pixel, the LPF (Low-Pass Filter) having coefficients of ⅛: 2/8: 2/8: 2/8:⅛ in the horizontal direction is applied to eliminate noise in the horizontal direction.

This is followed by the step of noise eliminator 402 in the vertical noise eliminator 402b applying a process of noise elimination in the vertical direction to the frame image data having been subjected to noise elimination in the horizontal direction. The vertical noise eliminator 402b applies the band eliminating filter to smoothen abrupt noise in the vertical direction. In the present embodiment, an attention pixel is extracted from the inputted frame image data in a prescribed sequence. Centering on this attention pixel, the LPF (Low-Pass Filter) having coefficients of ⅛: 2/8: 2/8: 2/8:⅛ in the vertical direction is applied to eliminate noise in the vertical direction. The noise eliminator 402 outputs to the motion vector detector 403 the frame image data whose noise has been eliminated in the aforementioned procedure.

The intermediate image generation section 14a, in the meantime, inputs the frame image data into the noise eliminator 402 from the previous frame image memory section 401. The noise eliminator 402 is provided with a horizontal noise eliminator 402c and vertical noise eliminator 402d, and serves eliminate the horizontal noise by means of the horizontal noise eliminator 402c. The method for removing the noise by means of the horizontal noise eliminator 402c is the same as that by means of the aforementioned horizontal noise eliminator 402a and the description will be omitted.

This is followed by the step of the vertical noise eliminator 402d eliminating vertical noise of the frame image data subjected to horizontal noise elimination. The resulting data is outputted to the motion vector detector 403. The method for removing the noise by means of the vertical noise eliminator 402d is the same as that by means of the aforementioned vertical noise eliminator 402b and the description will be omitted.

The motion vector detector 403 is provided with a comparison block size decision section 403a, search range size decision section 403b, comparison block motion processing section 403c, brightness difference decision section 403d, the minimum brightness difference position storage section 403e, undetected destination decision section 403f, interpolation method selection section 403g.

When the frame image data of the latest frame subjected to the aforementioned noise elimination process and the frame image data of the frame one frame before the latest one have been inputted, the motion vector detector 403 takes the following step to detect the motion vector, wherein the frame image data of the frame one frame before the latest one is used as an original frame and the latest frame image data is assumed as a reference frame:

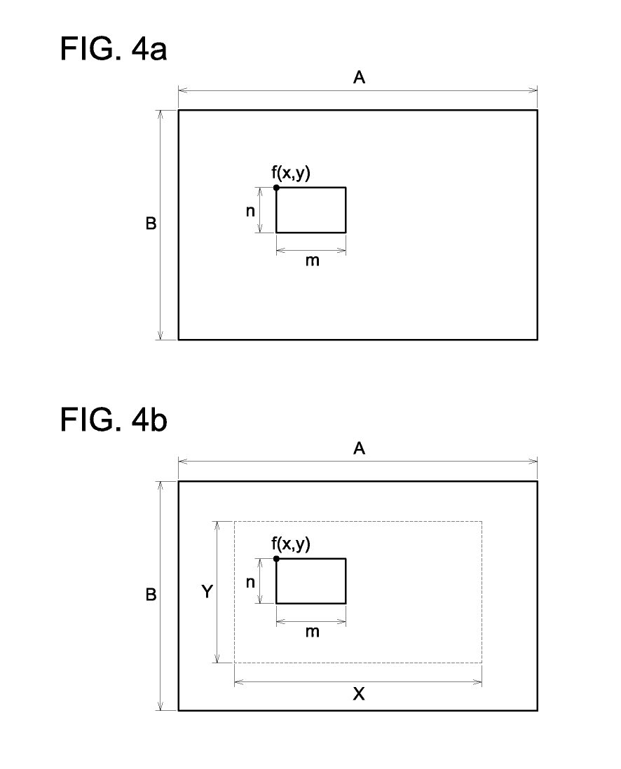

The motion vector detector 403 uses the comparison block size decision section 403a to split the original frame into a plurality of blocks each having the size of m×n. The motion vector detector 403 uses the search range size decision section 403b to ensure that the search range having a search range size of X×Y will be set on the reference frame based on the position in the original frame whose position is to be searched. To put it more specifically, for example, as shown in FIG. 4a, when the block whose position is to be searched is located at f(x, y), search range having a size of X×Y is set at the position shown in FIG. 4b, on the reference frame. If the frame image data has an image size of A×B and excessive processing load is applied by a search in the entire range of frame image data, set the search range size at X×Y (X<A, Y<B), for example. If the processing load is not excessive, the search range size can be set to X×Y (X=A, Y=B). It is also possible to arrange such a configuration that the search range size can be adjusted in conformity to the diagnostic position or settings.

The motion vector detector 403 allows the comparison block motion processing section 403c to scan the block whose position is to be searched, within the search range of the reference frame, and uses the brightness difference decision section 403d to search for the image having a high degree of correlativity with the image of this block on the reference frame, and to identify the destination of the image of this block.

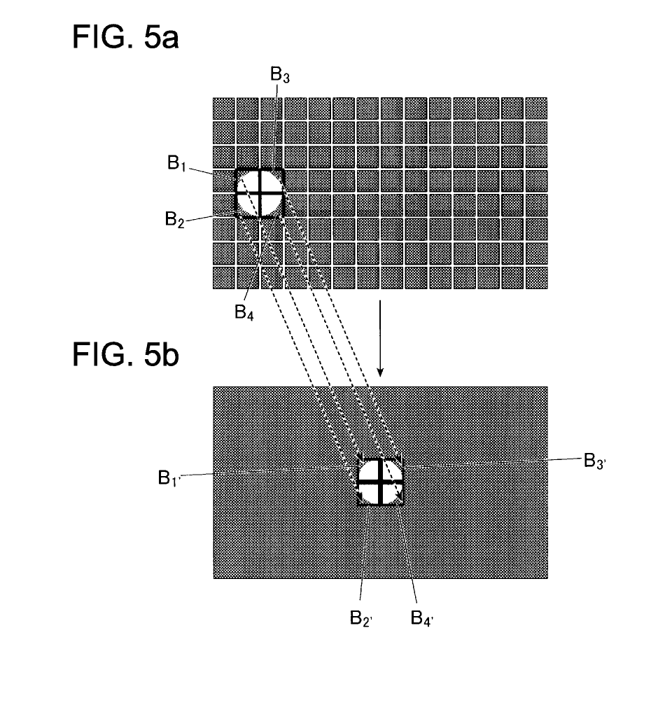

Referring to FIGS. 5a and 5b, the following describes how to identify the destination of the block image: In the example of FIGS. 5a and 5b, the size of each block of the original frame is assumed as 8×8 dots.

As shown in FIG. 5a, if the block whose position is to be searched for is the block B₁ of the original frame, the difference in brightness between and the image composed of the block B₁ and the image to be referenced in the reference frame having the same size as the block B₁ is checked by the brightness difference decision section 403d, while the block B₁ in the search range of the reference frame is scanned by the compared block motion processing section 403c. Then the block B₁′ as a destination of the block B₁ is identified, as shown in FIG. 5b by detecting the position wherein the brightness difference is the minimum, namely, the position of the greatest correlativity.

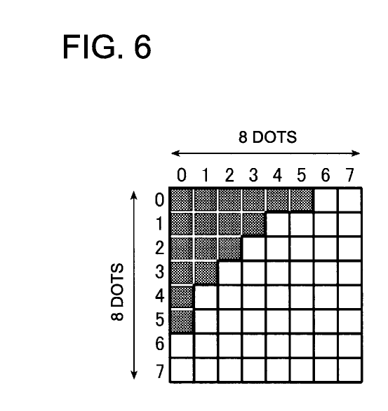

In the present embodiment, for example, the destination of the block B₁ is identified by determining the difference in brightness between 8×8=64 dots constituting the block B₁ and the 8×8=64 dots of the image to be referenced in the reference frame. The image of the block B₁ has a pixel structure as shown in FIG. 6. The absolute value of brightness difference is found out for each pixel and the sum total is calculated. This procedure is used to find out the brightness difference between the image constituting the block B₁ and the image to be referenced in the reference frame. The similar procedure is used to search the destinations of other blocks B₂, B₃, and B₄, and the blocks B₂′, B₃′, B₄′ are identified.

In the calculation of the brightness difference for each pixel, it is also possible to calculate only the brightness difference at specific positions, without the brightness difference being calculated for all of the 8×8 pixels. For example, it is also possible to calculate the brightness difference for the pixels on the upper left, lower left, upper right and lower right alone.

When the destination of the image whose position has been searched for by the aforementioned procedure has been identified, the motion vector detector 403 allows the information on the destination of that block to be stored in the minimum brightness difference position storage section 403e.

In the meantime, if there is any block for which the destination could not be found out as a result of detecting the destination for each block in the aforementioned manner, the motion vector detector 403 provides the undetected destination decision section 403f with the information that the destination could not be found out for that block.

As described above, after detection of the motion vector for each block, if the image of the portion to be generated in the generation of the intermediate image data (to be described later) is the image whose destination has been detected, the motion vector detector 403 allows the minimum brightness difference position storage section 403e to output the information to that effect to the interpolation method selection section 403g. At the same time, the motion vector detector 403 outputs the information indicating the destination to the intermediate data generation section for destination detection 405. In the meantime, if the image of the portion to be generated is the image whose destination has not been detected, the information to that effect is output to the interpolation method selection section 403g by the undetected destination decision section 403f. The motion vector detector 403 allows the interpolation method selection section 403g to output to the switching section 404 the signal in conformity to the inputted information from the minimum brightness difference position storage section 403e and undetected destination decision section 403f. To put it more specifically, if there is an input of information from the minimum brightness difference position storage section 403e, the interpolation method selection section 403g outputs the signal showing the intention of generating the intermediate image. If there is an input of information from the undetected destination decision section 403f, the interpolation method selection section 403g outputs the signal showing the intention of generating an averaging image.

The switching section 404 selects the position of the switch in conformity to the signal supplied from the interpolation method selection section 403g. To put it more specifically, when the signal of generating the intermediate image has been inputted from the interpolation method selection section 403g, the switching section 404 sets the switch to (M). When the signal of generating the averaging image has been inputted, the switching section 404 sets the switch to (A).

When the (M) has been selected by the switching section 404, the inputted frame image data of the latest frame and the frame image data of the frame one frame before the latest one stored in the previous frame image memory section 401 are inputted into the intermediate data generation section for destination detection 405.

The intermediate data generation section for destination detection 405 identifies the position of the image at the source as a target for generating the intermediate image from the frame image data of the preceding frame, and identifies the position of the image at the destination, based on the information showing the destination outputted from the minimum brightness difference position storage section 403e. The following formulae (1) and (2) are used to calculate the intermediate data generation position M1 (x, y). In the following formulae, P (x, y) indicates the reference position of the image at the source and F (x, y) denotes the reference position of the image at the destination.

M1(x)=P(x)+F(x)−P(x))/2 (1)

M1(y)=P(y)+F(y)−P(y))/2 (2)

Further, in the present embodiment, a plurality of pieces of intermediate image data are generated in conformity to particular requirements, as will be described later. The generation position Mm (x, y) of the intermediate data in the intermediate image data in this case is calculated from the following formulae (3) and (4). In the following formulae, “n” denotes the number of generated intermediate images, and “m” assumes the numeral in the range from 1 through “n”.

Mm(x)=P(x)+(F(x)−P(x))m/(n+1) (3)

Mm(y)=P(y)+(F(y)−P(y))m/(n+1) (4)

The intermediate data generation section for destination detection 405 generates intermediate data to ensure that the image at the source as a target for generating the intermediate data will be arranged in such a way that the generation position M1 (x, y) (or Mm (x, y)) of the intermediate data having been calculated is used as a reference position. The intermediate data generated by the aforementioned procedure is stored, for example, in the intermediate image data buffer (not illustrated).

When the (A) has been selected by the switching section 404, the image data of the block whose destination has not been detected is read from the frame image data of the preceding frame stored in the previous frame image memory section 401, and the data of the image arranged at the same position as the image data of the block whose destination has not been detected is read from the frame image data of the latest frame having been inputted. Then, these two pieces of image data are each inputted into the intermediate data generation section 406 for undetected destination mode.

After weights have been assigned to the inputted image data by the weighing sections 406a and 406b, the intermediate data generation section 406 for undetected destination mode allows the averaging image data to be generated through addition by the adder 406c. In present embodiment, each of the weighing factors of the weighing sections 406a and 406b is 0.5. These factors can be set to any desired value. The averaging image data generated by the adder 406c is stored in the aforementioned intermediate image data buffet.

When one frame of intermediate image data has been generated at the intermediate image data buffer in the aforementioned procedure, the intermediate image generation section 14a allows the image data to be sent to the memory section 15. In the aforementioned procedure, the intermediate image generation section 14a generates the preset number of pieces of intermediate image data for each frame, and these pieces of data are sequentially output to the memory section 15.

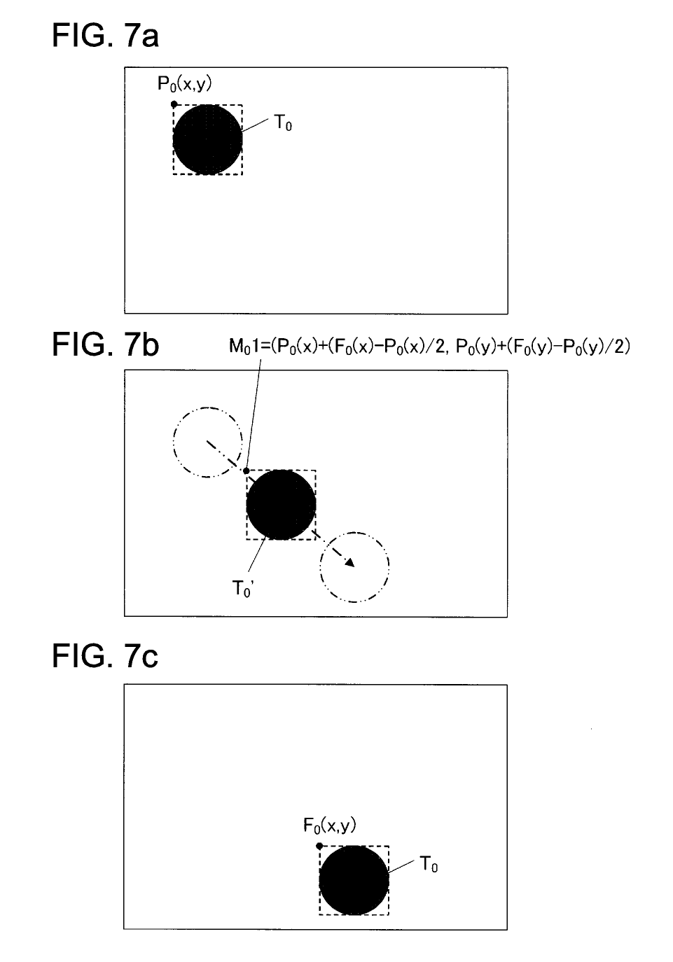

When the intermediate image data has been generated in the aforementioned procedure, the intermediate image data is inserted in chronological order between the pieces of frame image data generated for each frame based on the sound ray data, and is displayed on the display section 17. To put it more specifically, for example, as shown in FIGS. 7A and 7C, when the frame image data has been generated, the intermediate image shown in FIG. 7b will be generated. A moving subject T₀ is arranged with reference to the coordinate P₀ (x, y), in the frame image data of the preceding frame shown in FIG. 7a, One frame after, the moving subject T₀ moves to the position wherein the coordinate F₀ (x, y) is the reference position, as shown in FIG. 7c.

When the destination of the moving subject T₀ has been identified by the intermediate image generation section 14a in the aforementioned procedure, a step is taken to calculate the position intermediate between the moving subjects T₀ of two frames to generate the intermediate image data so that the moving subject T₀′ as the same image as the moving subject T₀ will be arranged, as shown in FIG. 7b, wherein the calculated position M₀1 (x, y) is used as a reference position. When the intermediate image data has been generated in the aforementioned procedure, a smooth movement of the moving subject can be displayed, even if the frame rate drops by transmit/receive of ultrasound waves.

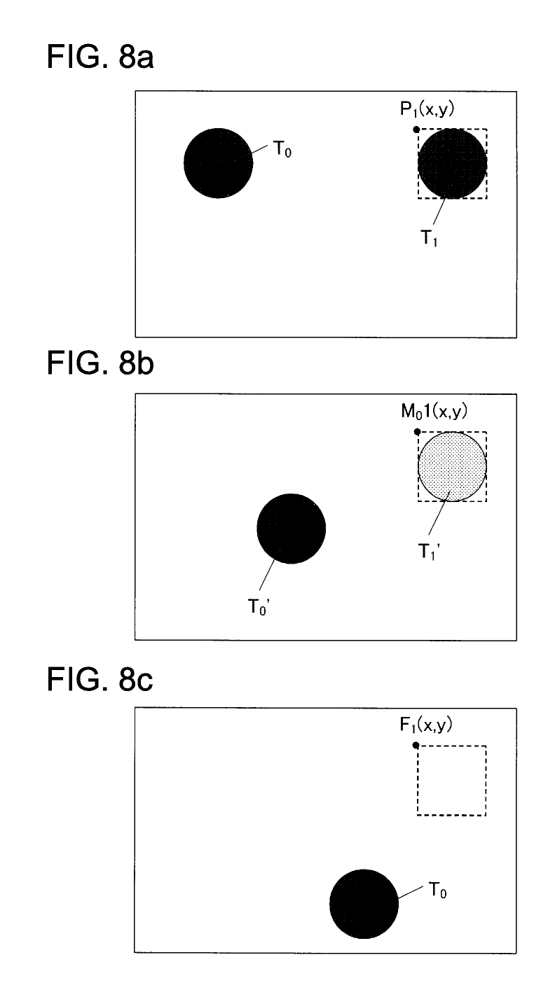

In the frame image data of the preceding frame shown in FIG. 8a, the moving subject T₁ in addition to the moving subject T₀ is arranged at P₁ (x, y). As shown in FIG. 8c, the moving subject T₁ is not present in the frame image data of the latest frame. Accordingly, the destination of the moving subject T1 is not detected by the motion vector detector 403 of the intermediate image generation section 14a. Here, in the intermediate image generation section 14a, the image data having the same image size located at the same reference position F₁ (x, y) as the image data constituting the moving subject T₁ in the frame image data of the preceding frame is extracted by the frame image data of the latest frame. The averaging image data with the moving subject T1′ arranged therein is generated at M₀1 (x, y) by the interpolation with the image data constituting the moving subject T₁. In this case, the reference position M₀1 (x, y) is the same coordinate as P₁ (x, y) and F1 (x, y). The residual image can be reduced by generation of the intermediate image data in the aforementioned procedure.

The averaging image data can be either the image data constituting the moving subject T1 in the frame image data of the preceding frame, or the image data in the latest frame image data having the same image size, located at the same reference position F₁ (x, y) as the image data constituting the moving subject T₁ in the frame image data of the preceding frame. The artifact can be reduced by generation of the intermediate image data in the aforementioned procedure.

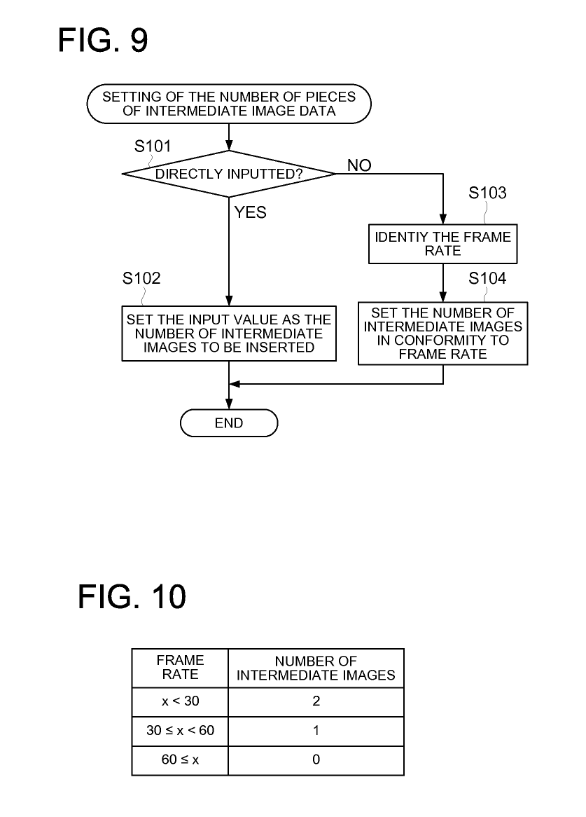

Referring to FIG. 9, the following describes the procedure of setting the number of the intermediate frames for the present embodiment. The procedure of setting the number of intermediate frames is implemented when a prescribed setting operation has been performed by the user.

The control section 18 checks whether or not the number of the frames to be inserted has been directly inputted by the operation of the operation input section 11 by the user (Step S101). When it has been determined that the number of the frames to be inserted has been directly inputted (Step S101: Y), the control section 18 sets the inputted values as the intermediate images to be inserted and displayed in chronological order in the display of the frame image (Step S102), terminating this processing.

When it has been determined that the number of the frames to be inserted has not directly been inputted (Step S101: N), the control section 18 identifies the frame rate by determining the currently preset ultrasound wave transmission/reception conditions (Step S103).

In this case, the frame rate depends on the settings of the ultrasound wave transmission/reception conditions, particularly on the number of the transmission beams to be applied for one frame, and the depth of the reflected ultrasound waves to be received.

The ultrasound diagnostic apparatus of recent years generally performs multi-stage focusing operation wherein a plurality of ultrasound wave transmissions and/or receptions are performed while the depth of the transmission focus point is varied in the same direction. When such a multi-stage focusing operation is performed, the scanning operations corresponding to the number of transmission focus points are performed for each frame. To be more specific, when an ultrasound probe with 192 transducers arranged thereon, 192 transmission beams are emitted in one scanning operation, for example. Further, for each frame, transmission beams are emitted in the number of times multiplied by the number of the transmission focus points. The time required for one transmission/reception of the ultrasound wave is increased in proportion to the depth of the transmission focus point. To avoid confusion between the transmission ultrasound waves and reflected ultrasound waves, a prescribed wait time must be provided for each emission of the transmission beam. Thus, as the number of the transmission focus points and the depth thereof are increased, a higher-quality ultrasound image can be obtained. However, this increases the time to get the image, and hence causes the frame rate to drop.

Some of the ultrasound diagnostic apparatuses of recent years are provided with a function of displaying the ultrasound image by THI (Tissue Harmonic Imaging). The THI outputs the fundamental wave and receives the secondary harmonic wave having the frequency twice that of the fundamental wave resulting from distortion of the fundamental wave in the subject. Based on this secondary harmonic wave, the ultrasound image is generated. Thus, the THI provides a technique of reducing the artifact. To generate the ultrasound image by THI, it is necessary to remove the fundamental wave contained in the reflected ultrasound waves. Thus, a step is taken to perform pulse inversion wherein the ultrasound waves having the waveform having a phase opposite to that of the fundamental wave is further outputted to erase the fundamental wave component. This requires a transmission/reception of ultrasound waves twice the aforementioned level, and hence results in a further drop of frame rate.

As described above, an ultrasound image may not be obtained at a prescribed frame rate, depending on the settings of the ultrasound wave transmission/reception, namely, the, ultrasound wave transmission/reception conditions. The frame rate in conformity to ultrasound wave transmission/reception conditions is set in advance by the control section 18 to ensure that an appropriate ultrasound image can be obtained.

The control section 18 sets a value conforming to the frame rate identified in Step S103, as the number of intermediate images to be inserted and displayed in chronological order on the frame image display (Step S104), whereby processing terminates. To put it more specifically, the Table shown in FIG. 10 is stored in the ROM. Referring to this Table, the control section 18 extracts and sets the number of intermediate images conforming to the frame rate. If the frame rate is below 30 F/s, for example, the number of intermediate images to be inserted is “2” by the control section 18. If the frame rate is 30 F/s or more but less than 60 F/s, the number of intermediate images to be inserted is “1”. If the frame rate is equal to or greater than 60 F/s, no intermediate image is inserted. The number of intermediate images to be inserted with respect to the frame rate can be set to a desired value. With consideration given to the processing speed in the generation of the intermediate image data, this number is preferably set to such a value that a residual image is not recognized by the user. Generally, when an intermediate image has been inserted, the residual image is not recognized very much if the frame rate is equal to or greater than 60 F/s. A residual image is hardly recognized if the frame rate is equal to or greater than 100 F/s,

In the present embodiment, the control section 18 serves as a freeze control section to provide freeze control in such a way that, when the freeze operation of the operation input section 11 has been performed, the ultrasound diagnosis image (including the frame image and intermediate image) displayed on the display section 17 at the moment the freeze operation has been received is kept in the state displayed on the display section 17.

In the present embodiment, the control section 18 serves as a display control section to provide frame feed display in such a way that, when the frame advance operation has been made on the operation input section 11, the ultrasound diagnosis images displayed on the display section 17 are converted in units of image data in chronological order and is displayed on the display section 17. The frame advance operation through the operation input section 11 includes the operation of displaying the frame image which is chronologically one frame before the frame image displayed on the display section 17, and the operation of displaying the frame image which is chronologically one frame after the frame image displayed on the display section 17.

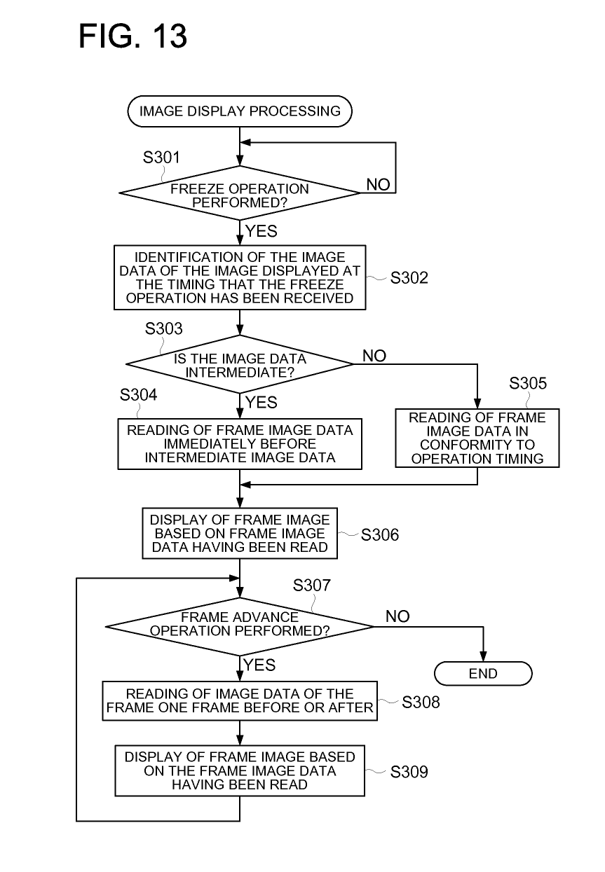

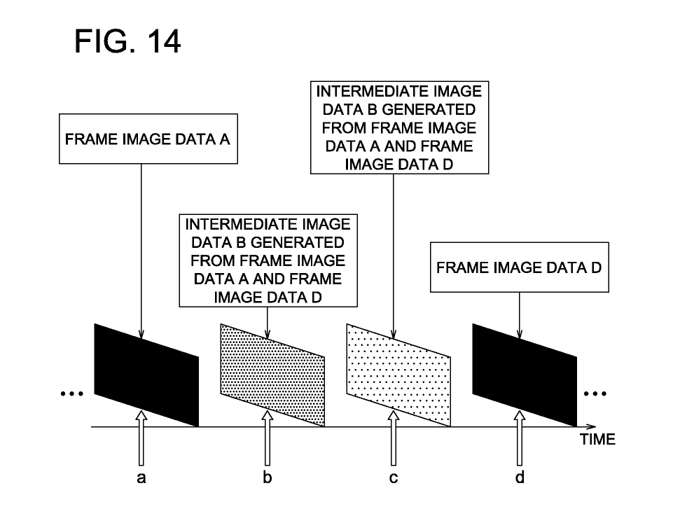

Referring to FIGS. 13 and 14, the following describes the image display processing executed by the control section 18 of the aforementioned configuration. The image display processing to be described below is executed in conformity to a prescribed operation by a doctor or radiographer in an examination by ultrasound diagnosis using an ultrasound diagnostic apparatus S of the present embodiment.

The control section 18 determines whether or not a freeze operation has been inputted from the operation input section 11 (Step S301).

When it is determined that the freeze operation has been inputted (Step S301: Y), the control section 18 takes a step of determining whether the data of the ultrasound diagnosis image displayed on the display section 17 at the moment the freeze operation has been received is the frame image data generated by the image generation section 14 or the intermediate image data generated by the intermediate image generation section 14a (Step S302).

As described above, when the image data is stored in the memory section 15, the control section 18 ensures that the frame image data can be identified from the intermediate image data. Based on the identification information added to the image data, the control section 18 determines if the image data conforming to the freeze operation is the frame image data generated by the image generation section 14, or the intermediate image data generated by the intermediate image generation section 14a.

As shown in FIG. 12b, when the image data is stored in the separate storage region inside the memory section 15, the control section 18 determines, based on the address of the storage region of the stored image data, if the image data conforming to the freeze operation is the frame image data generated by the image generation section 14, or intermediate image data generated by the intermediate image generation section 14a.

As shown in FIG. 12c, if the image data is stored in the separate memory sections 15a and 15b, the control section 18 determines, based on in which memory section the image data is stored, if the image data conforming to the freeze operation is the frame image data generated by the image generation section 14, or the intermediate image data generated by the intermediate image generation section 14a.

This is followed by the step of the control section 18 determining if the identified image data is the intermediate image data or not (Step S303). When it is determined that identified image data is the intermediate image data (Step S303: Y), the control section 18 reads out of the memory section 15 the frame image data of the frame image displayed on the display section 17 immediately before intermediate image based on the relevant intermediate image data (Step S304). In the meantime, if it is determined that identified image data is not the intermediate image data but the frame image data (Step S303: No), the control section 18 reads the relevant frame image data out of the memory section 15 (Step S305).

This is followed by the step of the control section 18 ensuring that the frame image conforming to the frame image data having read out is displayed on the display section 17 (Step S306).

Thus, as shown in FIG. 14, if the freeze operation has been received at the timing indicated by the arrow b or c when the intermediate image conforming to the intermediate image data B or C is displayed on the display section 17, the control section 18 ensures that the frame image conforming to the frame image data A displayed on the display section 17 immediately prior to this intermediate image is kept displayed on the display section 17. If the freeze operation has been received at the timing indicated by the arrow “a” when the frame image conforming to the frame image data A is displayed on the display section 17, the control section 18 ensures that the frame image conforming to this frame image data A is kept displayed on the display section 17. Similarly, if the freeze operation has been received at the timing indicated by the arrow “d” when the frame image conforming to the frame image data B is displayed on the display section 17, the control section 18 ensures that the frame image conforming to this frame image data D is kept displayed on the display section 17.

This is followed by the step of the control section 18 determining if the frame advance operation has been performed on the operation input section 11 or not (Step S307).

If it is determined that the frame advance operation has been performed (Step S307: Y), the control section 18, based on this frame advance operation, reads out of the memory section 15 the image data of the frame which is one frame before or after the frame image displayed on the display section 17 (Step S308). Then the control section 18 ensures that the frame image based on the image data of the frame which is one frame before or after the one having been read out is displayed on the display section 17 (Step S309). Then processing of the Step S307 is performed again.

The control section 18 repeats the procedures of Steps S307 through S309 until it is determined that the frame advance operation has not been performed in Step S307 (Step S308: N). If it has been determined that the frame advance operation is not performed, the control section 18 terminates the processing of image display.

After the freeze control has been started, the control section 18 terminates the freeze control in conformity to a lapse of a prescribed time or based on the freeze termination operation on the operation input section 11.

In this case, if the intermediate image data is generated in conformity to the frame image data, an artifact may occur to the intermediate image based on this intermediate image data. When such intermediate images are to be inserted into the frame image in chronological order and to be displayed as dynamic images on the ultrasound diagnosis image, an artifact does not raise any problem because it is invisible. However, if freeze control or frame feed display is implemented and an intermediate image containing an artifact is displayed as a still image, a diagnostic error may occur because the artifact is made visible to the user. To solve this problem, the aforementioned structure is adopted in the embodiment of the present invention.

As described above, the ultrasound probe 2 according to the present invention includes transducers 2a arranged in parallel that output transmission ultrasound waves toward a subject by means of a drive signal and, at the same time, output the reception signal by receiving the ultrasound waves reflected from the subject. The transmission section 12 supplies the transducers 2a with the drive signal. The receiving section 13 receives the reception signal sent from the transducers 2a. The image generation section 14 generates the frame image data obtained by converting the reception signal received by the receiving section 13, into the brightness information showing the brightness of the image. The intermediate image generation section 14a detects the moved image out of a plurality of pieces of image data in different frames, generated by the image generation section 14. Based on a plurality of pieces of frame image data, the intermediate image generation section 14a identifies the positions of the source and destination of the detected image. The intermediate image generation section 14a generates the intermediate image data wherein the moved image is arranged at the position obtained by interpolation of the positions of the source and destination of the identified image. The display section 17 displays the ultrasound diagnosis image, based on the frame image data generated by the image generation section 14 and the intermediate image data generated by the intermediate image generation section 14a. The display section 17 displays the ultrasound diagnosis image to ensure that the intermediate image based on the intermediate image data generated by the intermediate image generation section 14a will be inserted in chronological order into the image based on the frame image data generated by the image generation section 14 and then displayed. Thus, the interpolated image inserted in chronological order into the image based on the frame image data is displayed, whereby the display frame rate can be increased. This ensures occurrences of residual images to be reduced, even if the frame rate drops due to the ultrasound wave transmission/reception conditions. Further, this arrangement ensures smooth display of a dynamic image, and hence minimizes the possibility of the diagnostic target being overlooked, with the result that the precision of the ultrasound diagnosis is enhanced.

According to the embodiment of the present invention, intermediate image generation section 14a generates the intermediate image data to ensure that the moved image will be arranged at the point intermediate between the source and destination of the identified image. This provides accurate information on the position of the moved image among the interpolated image, and therefore, ensures a smoother display of the dynamic image.

According to the embodiment of the present invention, when the source or destination of the moved image cannot be identified, the intermediate image generation section 14a smoothes the image data of the moved image portion in the frame image data of the moved image whose position has been identified in a plurality of pieces of frame image data, and the image data of the image portion at the same position as the moved image in the frame image data of the moved image whose position is not identified. Then, the intermediate image generation section 14a generates the intermediate image data so that the smoothed image will be arranged in the same position as the moved image. This results in a gradual increase or decrease of the image brightness, and therefore, flickering of the image can be reduced.

According to the embodiment of the present invention, when the source and destination of the moved image cannot be identified, the intermediate image generation section 14a generates the intermediate image data to ensure that the image located at the same position as the moved image in the frame image data of the moved image whose position has been identified in a plurality of pieces of frame image data, or the moved image in the frame image data of the moved image whose position has not been identified will be arranged at the same position as the moved image. This arrangement reduces the artifact on the display of the image whose position of the source or destination cannot be identified.

According to the embodiment of the present invention, in conformity to the frame rate determined by the preset ultrasound wave transmission/reception conditions, the control section 18 sets the number of intermediate images to be displayed between frames. The intermediate image generation section 14a ensures that pieces of the intermediate image data to be displayed by insertion into the image based on the frame image data generated by the image generation section 14 are generated for the number of intermediate images set by the control section 18. This arrangement allows the appropriate number of pieces of the intermediate image data to be produced for the frame rate, and provides a smooth image display in conformity to the frame rate.

According to the embodiment of the present invention, in conformity to the operation of the operation input section 11, the control section 18 sets the number of intermediate images to be displayed between frames. The intermediate image generation section 14a ensures that pieces of the intermediate image data to be displayed by insertion into the image based on the frame image data generated by the image generation section 14 are generated for the number of intermediate images set by the control section 18. Thus, the number of intermediate images can be set in conformity to the diagnostic position or taste of the user, for example, with the result that smooth image display in response to user requirements is provided.

The display section 17 uses a liquid crystal display panel or organic EL display panel to display an ultrasound diagnosis image. When the present embodiment is applied to the liquid crystal display panel or organic EL display panel, a remarkable residual image suppression effect is ensured.

According to the embodiment of the present invention, when the ultrasound diagnosis image in conformity to the timing that the freeze operation has been received is the intermediate image based on the intermediate image data generated by the intermediate image generation section 14a, the control section 18 allows the frame image based on the frame image data to be displayed. Thus, even if an artifact occurs on the intermediate image at the time of generation of the intermediate image, diagnosis can be performed only by the frame image generated by the image generation section 14, whereby diagnostic error can be prevented.

According to the embodiment of the present invention, when the ultrasound diagnosis image in conformity to the timing that the freeze operation has been received is the intermediate image based on the intermediate image data generated by the intermediate image generation section 14a, the control section 18 displays the frame image to be displayed on the display section 17 immediately before or after the intermediate image in chronological order. This arrangement permits display of the frame image at the timing closest to the timing that the freeze operation has been performed, and ensures more accurate diagnosis.

According to the embodiment of the present invention, when the frame advance operation has been received, the control section 18 allows only the frame image based on the frame image data generated by the image generation section 14 to be displayed on the display section 17 by switching in image data units. Thus, even if an artifact occurs on the intermediate image at the time of generation of the intermediate image, diagnosis can be performed only by the frame image generated by the image generation section 14, whereby diagnostic error can be prevented.

The description of the embodiment in the present invention shows only an example of the ultrasound diagnostic apparatus in the present invention, without the present invention being restricted thereto. The details of the structure and operation of various functional components of the ultrasound diagnostic apparatus can be modified as appropriate.

In the embodiment of the present invention, in the detection of the motion vector by the motion vector detector 403, the motion vector is detected on the assumption that the original frame is the frame image data of the frame one frame before the latest one, and the reference frame is the frame image data of the latest frame. However, it is also possible to arrange such a configuration that the motion vector is detected on the assumption that the original frame is the frame image of the latest frame, and the reference frame is the frame image data of the frame latest one frame before the latest one.

In the embodiment of the present invention, the number of intermediate frames can be set by the user. However, the number of intermediate frames can be fixed. In the present embodiment, the number of intermediate frames can be set by choosing between inputting a desired value and inputting a specified value according to frame rate. However, these functions can be restricted to any one of them.

In the embodiment of the present invention, when the ultrasound diagnosis image displayed on the display section at the moment the freeze operation has been received is an intermediate image, the frame image to be displayed on the display section immediately before this intermediate image will be displayed. However, the frame image to be displayed on the display section immediately after this intermediate image can be allowed to be displayed.

In the embodiment of the present invention, the frame feed display is executed during the freeze control. However, the frame feed display can be executed when freeze control is not used.

In the embodiment of the present invention, in the frame feed display, what is displayed every time the frame advance operation is received is the frame image of the frame one frame before or after the frame image displayed on the display section. It is possible to display the frame image by switching pieces of image data in chronological order after a lapse of a prescribed time.

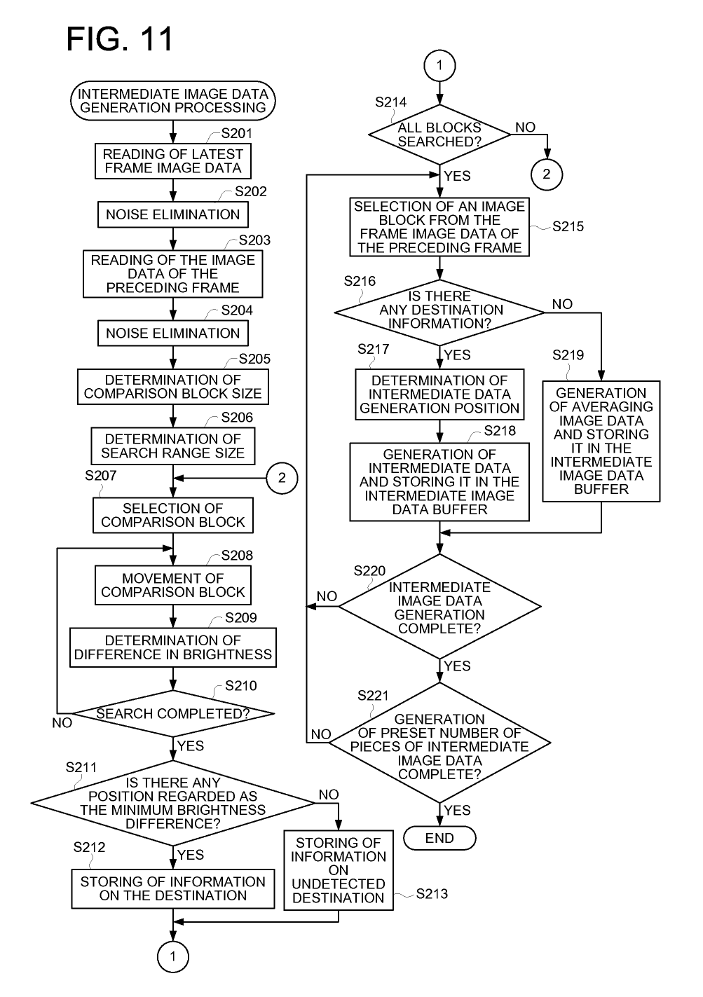

In the embodiment of the present invention, the intermediate image data is generated by the intermediate image generation section 14a. However, it is also possible to arrange such a configuration that the control section 18 serves as an intermediate image generation section and the memory section 15 is used as a work region, whereby the intermediate image data is generated by software control. For example, this is achieved by performing the intermediate image data processing as shown in FIG. 11.

The control section 18 reads the frame image data of the latest frame from the buffer of the memory section 15 that contains the frame image data of the latest frame (Step S201). The control section 18 then executes noise elimination processing, and applies the aforementioned processing of horizontal and vertical noise elimination to the frame image data having been read out (Step S202). From the buffer of the memory section 15 that contains the frame image data of the preceding frame, the control section 18 reads out the frame image data of the preceding frame (Step S203). The control section 18 then executes noise elimination processing, and applies the aforementioned processing of horizontal and vertical noise elimination to the frame image data having been read out (Step S204).

After noise elimination, the frame image data of the preceding frame is used as the original frame. By the aforementioned procedure, this data is split by the control section 18 into a plurality of blocks wherein the size is “m×n” (Step S205). The control section 18 determines the search range size in the aforementioned procedure, wherein the frame image data of the latest frame is used as a reference frame (Step S206). Out of a plurality of split blocks, the control section 18 selects the block for comparison in a prescribed sequence (Step S207). The control section 18 moves the selected block for comparison within the search range of the frame image data of the latest frame (Step S208), and determines the difference in brightness in the aforementioned procedure (Step S209).

The control section 18 then determines for all blocks if search has been completed or not (Step S210). When it is determined that search has been completed (Step S210: Y), a step is taken to determine whether or not there is any position regarded as the minimum brightness difference (Step S211). In the meantime, when it is determined that search has not been completed (Step S210: N), the control section 18 goes to the Step S208.

If it has been determined in Step S211 that there is a position regarded as the minimum brightness difference, i.e., a position of the greatest correlativity (Step S211: Y), the control section 18 stores in the RAM the position of the greatest correlativity (Step S212). In the meantime, if it has been determined that there is no position regarded as the minimum brightness difference, i.e., no destination of the image has been detected (Step S211: N), the control section 18 stores in the RAM the information that no destination has been detected (Step S213).

The control section 18 determines for all blocks if the search has been completed or not (Step S214). When it is determined that search has not been completed for all blocks (Step S210: N), the control section 18 executes processing of the Step S207. To select the next block to be searched, the control section 18 searches for the image destination in the aforementioned procedure.

When it is determined that search has been completed for all blocks (Step S214: Y), the control section 18 reads from the memory section 15 the frame image data of the preceding frame and splits a plurality of image blocks into the block size determined in Step S205. One of these blocks is then selected (Step S215).

The control section 18 determines whether or not there is any destination information on the image constituting the selected image block (Step S216). If it has been determined that there is destination information (Step S216: Y), the control section 18 works out the intermediate data generation position by interpolation calculation in the aforementioned procedure, based on the position of the source in the frame image data of the preceding frame and the position of the destination in the frame image data of the latest frame (Step S217). The control section 18 generates the intermediate data in the aforementioned procedure so that the image of the source will be arranged using the calculated intermediate data generation position as a reference position. This intermediate data is stored in the intermediate image data buffer of the memory section 15 (Step S218).

If it has been determined that there is no destination information, i.e., if the information on undetected destination corresponding to the selected image block is stored in the RAM (Step S216: N), the control section 18 uses the aforementioned procedure to extract the image data of the block whose destination was not detected, from the frame image data of the preceding frame. From the frame image data of the latest frame, the control section 18 extracts the image data of the image arranged at the same position as the image data of the block whose destination was not detected. These pieces of image data having been extracted are then assigned weights, and the results are added together. The control section 18 generates averaging image data so that it is arranged at the same position as the image data of the block whose destination was not detected. This data is stored in the intermediate image data buffer of the memory section 15 (Step S219).

For all the blocks in the frame image data of the preceding frame, the control section 18 determines whether or not intermediate data and averaging image data have been generated, and the generation of the intermediate image data is completed (Step S220). If it has been determined that the generation of the intermediate image data is completed, (Step S220: Y), the control section 18 goes to Step S221. If it has been determined that the generation of the intermediate image data is not completed (Step S220: N), the control section 18 goes to Step S215 and applies the aforementioned processing to other image blocks.

The control section 18 determines whether or not the number of the pieces of intermediate image data preset in the aforementioned process of setting the number of the pieces of the intermediate image data has been generated (Step S221). If it is determined that the preset number of the pieces of intermediate image data has been generated (Step S221: Y), processing terminates. If it is determined that the preset number of the pieces of intermediate image data has not been generated (Step S221: N), the control section 18 goes to Step S215 to further generate intermediate image data.

The same advantages as those of the embodiment of the present invention are also provided by executing the aforementioned processing.

In the above description, the entire frame image data is split into a plurality of blocks and destination search is conducted for each block. However, it is also possible to arrange such a configuration that part of the region in the frame image data is specified and destination search is conducted only for the image within the specified range. Further, based on a plurality of pieces of frame image data, a motion test is conducted to check for the moved portion of an image. A destination search can be conducted only on the images whose motion has been detected.

In the above description, for the moved images, the intermediate position of the arranged images in the aforementioned frame image data is acquired by interpolation from the frame image data of the latest frame and the frame image data of the frame one frame before the latest one. Intermediate image data (interpolated image data) is generated so that the moved images are arranged at the acquired positions, and is interpolated between the images displayed based on these pieces of frame image data. Then the image based on the intermediate image data is displayed. However, it is also possible to arrange such a configuration that the subsequent destination of the moving image (e.g., the image destination 0.5 frame alter the latest frame) is predicted by acquiring the position of the moved image from such frame image data by extrapolation, and extrapolated image data is generated to ensure that the moved image is arranged at the position acquired by extrapolation. Thus, the image based on extrapolated image data is displayed by extrapolation after each image based on such frame image data has been displayed. To put it another way, for display, an image based on the extrapolated image data generated in the aforementioned procedure can be inserted between the image displayed based on the frame image data of the latest frame and the image displayed based on the frame image data of the frame one frame after the latest one.

In the present embodiment, a computer-readable medium of the program in the present invention has been disclosed as an example of using a hard disk or nonvolatile memory such as a semiconductor, without the present invention being restricted thereto. Other examples of computer-readable medium that can be employed include a portable recording medium such as a CD-ROM. Further, a carrier wave is an example of appropriate medium whereby the data of the program in the present invention is provided through a communication line.

Thus, the present embodiment provides display of an image of smooth motion even if a frame rate drops, while suppressing generation of a residual image.

What is claimed is:

1. An ultrasound diagnostic apparatus comprising:
an ultrasound probe including a transducer which outputs an ultrasound wave toward a subject by means of a drive signal and outputs a reception signal by receiving an ultrasound wave reflected from the subject;
a transmission section which supplies the transducer with the drive signal;
a receiving section which receives the reception signal sent from the transducer;
an image generation section which generates frame image data obtained by converting the reception signal received by the receiving section into brightness information showing brightness of image;
an intermediate image generation section which detects a moved image out of a plurality of frame image data in different frames, generated by the image generation section, identifies a move source and a move destination of the detected image based on the plurality of frame image data, and generates intermediate image data so as to allocate the moved image at a position obtained by intermediating the identified move source and the identified move destination of the detected image; and
a display section which displays the ultrasound diagnosis image, based on the frame image data generated by the image generation section and the intermediate image data generated by the intermediate image generation section,
wherein the display section displays the ultrasound diagnosis image so that an intermediate image based on the intermediate image data generated by the intermediate image generation section is inserted in a chronological order into an image based on the frame image data generated by the image generation section.

2. The ultrasound diagnostic apparatus described in claim 1, wherein the intermediate image generation section generates the intermediate image data so as to allocate the moved image at a middle point between the move source and the move destination of the detected image.

3. The ultrasound diagnostic apparatus described in claim 1, wherein, when the move source or the move destination of the moved image cannot be identified, the intermediate image generation section generates the intermediate image data such that, in the plurality of frame image data, image data of a moved image portion in the frame image data of the moved image whose position has been identified and image data of an image portion at the same position as the moved image in frame image data of the moved image whose position is not identified are smoothed and the smoothed image is allocated in the same position as the moved image.

4. The ultrasound diagnostic apparatus described in claim 1, wherein, when the move source or the move destination of the moved image cannot be identified, the intermediate image generation section generates the intermediate image data so that, in the plurality of frame image data, the moved image in frame image data of which position of the moved image has been identified or image at the same position as the moved image in frame image data of which position of the moved image is not identified, is allocated in the same position as the moved image.

5. The ultrasound diagnostic apparatus described in claim 1, further comprising a control section setting a number of intermediate images to be displayed between frames in conformity to a frame rate determined by a preset ultrasound wave transmission/reception condition,
wherein the intermediate image generation section generates the intermediate image data to be inserted into image based on the frame image data generated by the image generation section and displayed, by the number of intermediate images set by the control section.

6. The ultrasound diagnostic apparatus described in claim 1, further comprising: an operation input section capable of accepting an operation by a user; and a control section setting a number of intermediate image to be displayed between frames in conformity to the operation to the operation input section,
wherein the intermediate image generation section generates the intermediate image data to be inserted into image based on the frame image data generated by the image generation section and displayed, by the number of intermediate image set by the control section.

7. The ultrasound diagnostic apparatus described in claim 1, wherein the display section uses a liquid crystal display panel or organic EL display panel to display the ultrasound diagnosis image.

8. The ultrasound diagnostic apparatus described in claim 1, further comprising a freeze control section which, when receiving a freeze operation, performs a freeze control to maintain the ultrasound diagnosis image in a state of being displayed on the display section, in conformity to the timing when receiving the freeze operation,
wherein, in conformity to the timing when receiving the freeze operation, the freeze control section does not display the intermediate image based on the intermediate image data on the display section but displays only the image based on the frame image data, on the display section.

9. The ultrasound diagnostic apparatus described in claim 1, further comprising a freeze control section which, when receiving a freeze operation, performs a freeze control to maintain the ultrasound diagnosis image in a state of being displayed on the display section in conformity to the timing when receiving the freeze operation,
wherein, in a case when the ultrasound diagnosis image in conformity to the timing when receiving the freeze operation is the intermediate image based on the intermediate image data generated by the intermediate image generation section, the freeze control section displays the image based on the frame image data, on the display section.

10. The ultrasound diagnostic apparatus described in claim 9, wherein, in a case when the ultrasound diagnosis image in conformity to the timing when receiving the freeze operation is the intermediate image based on the intermediate image data generated by the intermediate image generation section, the control section displays the frame image immediately before or after the intermediate image in chronological order, on the display section.

11. The ultrasound diagnostic apparatus described in claim 1, further comprising a display control section which provides a frame by frame advance display of the ultrasound diagnosis image in chronological order on the display section by switching in image data units, when receiving a frame by frame advance operation,
wherein the display control section provides the frame by frame advance display only of the frame image based on the frame image data generated by the image generation section on the display section.

12. A computer-readable recording medium storing a program to be executed by a computer provided in an ultrasound diagnosis apparatus comprising an ultrasound probe including a transducer which outputs ultrasound waves toward a subject by means of a drive signal and outputs a reception signal by receiving ultrasound waves reflected from the subject, wherein the program makes the computer function as:
a transmission section which supplies the transducer with a drive signal;
a receiving section which receives the reception signal output from the transducer;
an image generation section which generates frame image data obtained by converting the reception signal received by the receiving section, into brightness information showing a brightness of the image;
an intermediate image generation section which detects moved image out of a plurality of frame image data in different frames, generated by the image generation section and, based on the plurality of frame image data, identifies a move source and a move destination of the detected image, and generates intermediate image data which allocates the moved image at a position obtained by interpolating the move source and the move destination of the detected image; and
a display section which displays the ultrasound diagnosis image, based on the frame image data generated by the image generation section and the intermediate image data generated by the intermediate image generation section,
wherein the ultrasound diagnosis image is displayed on the display section so that intermediate image based on the intermediate image data generated by the intermediate image generation section is inserted in a chronological order into image based on the frame image data generated by the image generation section.
